(19) United States
(12) Patent Application Publication
(10) Pub. No.: US 2010/0173347 A1
(43) Pub. Date: Jul. 8, 2010

(54) STABILIZED GOLD NANOPARTICLES AND METHODS OF MAKING THE SAME

(76) Inventors: Michael A. Brook, Ancaster (CA); Yingfu Li, Dundas (CA); Ferdinand Gonzaga, Hamilton (CA); Weian Zhao, Hamilton (CA); Monsur M. Ali, Hamilton (CA); Sergio D. Aguirre, Bolton (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/593,719

(22) PCT Filed: Apr. 2, 2008
(86) PCT No.: PCT/CA08/00611

§ 371 Date: Mar. 9, 2010

Related U.S. Application Data
(60) Provisional application No. 60/909,632, filed on Apr. 2, 2007.

(51) Int. Cl. C12Q 1/02; C07H 21/02; C07F 1/12; G01N 21/00; C12Q 1/00; C07H 21/04

(52) U.S. Cl. 435/29; 536/23.1; 556/110; 436/164; 435/4

(57) ABSTRACT

The present disclosure relates to water-soluble stable gold nanoparticles (AuNPs) and methods for making the same. The present disclosure also includes the use of AuNPs, for example, in biological, medical and environmental assays for the detection of analytes, as well as biological and medical imaging.

### FIELD OF THE DISCLOSURE

The present disclosure relates to water-dispersible stable gold nanoparticles (AuNPs) and methods for making the same. The present disclosure also includes the use of AuNPs in biological, medical and environmental assays, including paper-based assays, for the detection of analytes, as well as imaging.

### BACKGROUND

AuNPs (AuNPs) have unique physical properties (e.g. surface plasmon resonance (SPR)) that are tuned by nanoparticle size, shape and surface functionalities. These unique properties make AuNPs highly suitable building blocks for constructing nano-materials and, for example, as reporters of biological processes.

The reduction of aqueous gold salts is the most widely used method of preparing AuNPs in solution. Introducing agents (such as thiols, amines, phosphines, polymers and surfactants) during synthesis provides an exceptional degree of morphological and size control in the preparation of AuNPs. For example, see Brust. M, et al. J. Chem. Soc., Chem. Commun. 801 (1994). The surface functionalities and surface properties (hydrophilicity) are introduced at the same time.

AuNPs have recently been used as reporters for the detection of various substances such as DNA (see Elghanian, R. et al, Science, 277, 1078 (1997); Mirkin C, et al. U.S. Pat. No. 6,361,944, 2002), proteins (see Huang, C. et al, Anal. Chem. 77, 5735 (2005)) and metal ions (see Liu, J. et al, J. Am. Chem. Soc. 125, 6642 (2003)). The principle of AuNP biosensors relies on the unique SPR of AuNPs, that is, the well-dispersed AuNP appears red in color whereas the aggregated AuNPs have a blue (or purple) color. A target analyte or a biological process that triggers (directly or indirectly) AuNP aggregation (or redispersion of aggregate) can in principle be detected by color changes. As the interparticle plasmon coupling yields a huge absorption band shift (up to 300 nm), the color change can be observed by the naked eye and therefore no sophisticated instruments are required. Quantitative analysis can be realized by recording the absorption spectra (normally at an arbitrarily chosen assay time given the fact that AuNP aggregation is a dynamic and continuous process) using a standard spectrophotometer.

AuNP aggregation can be induced by an “interparticle crosslinking” mechanism that uses the target analyte as a crosslinker to bridge biomolecule receptor modified AuNPs into aggregates. For example, Mirkin and coworkers extensively studied the DNA-induced interparticle crosslinking system and its use for the detection of DNA (see Rosi, N. L, et al. Chem. Rev. 105, 1547 (2005)).

To achieve the sensitivity and specificity of a AuNP biosensor, the attachment of biological receptors onto AuNP surface is generally required. The attachment of functional nucleic acid receptors (aptamers, DNA enzymes and ribozymes, etc) onto AuNP surfaces provides opportunities for developing generic, simple and rapid AuNP biosensors for sensitive and specific detection of biological events.

Most of the previous assays using AuNPs as colorimetric signal indictors are performed in a solution-phase. However, this may not be suitable for practical applications such as home and clinical tests. Efforts have therefore been made to develop more user-friendly test kits. Particularly, the lateral flow based dipstick tests using the intense color of AuNPs for detection of DNA (see Glynou, et al. Anal. Chem. 2003, 75, 4155; Litos, et al. Anal. Chem. 2007, 79, 395), and AuNP color change during lateral flow for the detection of adenosine and cocaine (see Liu, et al. Angew. Chem. 2006, 118, 8123) have been developed. Nevertheless, these devices always involve rather complicated components such as wicking pads, conjugate pads, polymer membranes, absorbent pads and plastic adhesive backing, which may limit wide and practical use. In addition, additional biological functionalization steps (such as conjugation of streptavidin) of these devices are often required in order to trap the AuNP probes, which represent further limitations of such assays.

Paper, a web formed from cellulose fibers, optionally containing lignin, is an inexpensive high surface area support, the structure of which is highly controllable. The surface nature of the material is readily modified to be more hydrophobic (e.g. for printing, by use of sizing agents), stronger (by addition of wet and dry strength polymers), brighter or colored by the addition of pigments, and made more opaque and stronger by the addition of mineral fillers (e.g. clay, silica). Paper is widely used as a filtration aid to separate materials, and as a medium to carry information (e.g. by printing of ink on paper).

Martinez has demonstrated the possibility of using paper as a platform for bioassays (Martinez, et al. Angew. Chem. 2007, 119, 1340). Here, dye molecules (which have much lower extinction coefficients than AuNPs) were responsible for the color changes that were modulated by enzymatic reactions.

### SUMMARY

The present disclosure relates to monodisperse water-soluble highly stabilized AuNPs that have tunable sizes and shapes. The present disclosure also relates to a method of making the AuNPs. Further, the present disclosure relates to the use of AuNPs in biological, medical and environmental assays, including paper-based assays, for the detection of analytes, as well as imaging.

Accordingly, the present disclosure includes a water-miscible AuNP, wherein the AuNP is stabilized with at least one capping ligand, the capping ligand having a AuNP binding domain and a charged domain. In one embodiment the AuNP has a diameter of about 1 nm to about 100 nm. In a further embodiment, the diameter of the AuNP is about 1 nm to about 10 nm. In a subsequent embodiment, the diameter of the AuNP is about 2 nm to about 5 nm. In a further embodiment the AuNPs of the present disclosure are monodisperse.

In an embodiment of the disclosure, the charged domain on the capping ligand is a negatively or positively charged moiety that serves to repel the nanoparticles from each other to inhibit association or aggregation of the particles, in particular at high salt concentrations.

In another embodiment of the present disclosure, the capping ligand is a nucleotide, a deoxynucleotide, a functionalized nucleotide, a nucleoside, an oligonucleotide, a functionalized oligonucleotide, a nucleic acid polymer, a thiol or an amine. In a further embodiment, the nucleotide is adenosine 5′-triphosphate (ATP), adenosine 5′-diphosphate (ADP), adenosine 5′-monophosphate (AMP), guanosine 5′-triphosphate (GTP), cytidine 5′-triphosphate (CTP), thymidine 5′-triphosphate (TTP), inosine 5′-triphosphate or uracil 5′-triphosphate. In another embodiment, the nucleotide is adenosine 5′-triphosphate.

In another embodiment of the disclosure, the oligonucleotide is a DNA or RNA oligonucleotide. In a further embodiment, the DNA or RNA oligonucleotide is an aptamer that can bind to a target. In another embodiment, the target is a protein, an enzyme, nucleic acid, a small molecule, a metal ion, a bacteria or a pathogen.

In an embodiment of the disclosure, the capping ligand is further functionalized. In another embodiment, the capping ligand is chemically functionalized or enzymatically functionalized. In a further embodiment, the functionalization of the capping ligand allows the attachment of recognition molecules or entities to the particle.

In another embodiment of the disclosure, when the capping ligand of the AuNP is displaced, the AuNPs form aggregates.

In further embodiments of the disclosure there is included a method for the production of water-miscible AuNPs. In an embodiment, the method comprises reacting a solution of a gold salt in a suitable solvent with a stabilizing capping ligand and a reducing agent, wherein the capping ligand has a binding domain and a charged domain.

In another embodiment, the gold salt is HAuCl₄. In a further embodiment, the HAuCl₄ is present in the suitable solvent in an amount of about 100 μM to about 100 mM. In another embodiment, the HAuClₐ is present in the suitable solvent in an amount of about 100 μM to about 10 mM. In a further embodiment, the solvent is water.

In an embodiment of the disclosure, the charged domain on the capping ligand is a negatively or positively charged moiety that serves to repel the nanoparticles from each other to inhibit association or aggregation of the particles, in particular at high salt concentrations.

In another embodiment of the present disclosure, the stabilizing capping ligand is a nucleotide. In another embodiment, the nucleotide is adenosine 5′-triphosphate (ATP), adenosine 5′-diphosphate (ADP), adenosine 5′-monophosphate (AMP), adenosine, guanosine 5′-triphosphate (GTP), cytidine 5′-triphosphate (CTP), thymidine 5′-triphosphate (GTP), inosine 5′-triphosphate or uracil 5′-triphosphate. In a further embodiment, the nucleotide is adenosine 5′-triphosphate (ATP).

In an embodiment of the disclosure, the stabilizing capping ligand is present in an amount of about 100 μM to about 100 mM. In a further embodiment, the stabilizing capping ligand is present in an amount of about 100 μM to about 10 mM.

In another embodiment of the present disclosure, the reducing agent is a hydride reducing agent, such as sodium borohydride (NaBH₄). In a further embodiment, the sodium borohydride is present in an amount of about 100 μM to about 500 mM. In another embodiment, the sodium borohydride is present in an amount of about 100 μM to about 100 mM.

In an embodiment of the present disclosure, the reaction is performed at a temperature of about 10° C. to about 50° C. In another embodiment, the reaction is performed at about room temperature. In a subsequent embodiment, the reaction is performed for a period of about 1 hour to about 5 hours. In a further embodiment, the reaction is performed for about 3 hours.

In another embodiment of the present disclosure, the molar ratio of the gold salt to the capping ligand ([gold salt]:[capping ligand]) is about 0.1 to about 10. In another embodiment, the molar ratio of the reducing agent to the gold salt ([reducing agent]:[gold salt]) is about 15 to about 20. In another embodiment, the size of the stable AuNPs is controlled by the molar ratios of the gold salt, the capping ligand and the reducing agent.

In further embodiments of the present disclosure, the AuNPs are useful for biological, medical and environmental assays, including paper-based assays, and for the detection of analytes, as well as for imaging, such as biological or medical imaging. Any process that induces AuNP aggregation, or dissociation of AuNP aggregates, can be monitored or detected using the assays described herein.

Accordingly, in an embodiment of the present disclosure, there is included a method of monitoring or detecting a substance or process that induces aggregation of AuNPs, or dissociation of AuNP aggregates, comprising contacting the process or substance with the AuNPs of the present disclosure and observing or detecting a color change due to the aggregation or dissociation of the particles, wherein a color change is indicative of the substance or process.

In an embodiment, the AuNPs of the present disclosure are useful for the detection of an analyte. In an embodiment, the analyte is a protein, an enzyme, nucleic acid, small molecule, metal ions, bacteria or pathogen.

In a further embodiment, the AuNPs of the present disclosure are useful for the labeling or imaging of biological or medical samples.

In another embodiment, the AuNPs of the present disclosure are useful for a method of conducting a biological, medical or environmental assay. Accordingly, the present disclosure includes a method of determining the presence or absence of an analyte comprising:
a) providing a solution of the AuNPs, or aggregates of AuNPs, of the present disclosure;
b) mixing the solution of AuNPs or aggregates with a biological, medical or environmental sample comprising an analyte; and
c) determining the presence or absence of the analyte.
   In an embodiment, the presence or absence of the analyte in the solution is qualitatively determined by a color change of the solution, the color change being the result of aggregation of the particles, or dissociation of aggregated particles, caused by interaction with the analyte. In a subsequent embodiment, the presence or absence of the analyte in the solution is quantitatively determined by ultraviolet or visible light spectroscopy. In another embodiment, the analyte is a protein, an enzyme, nucleic acid, a small molecule, a metal ion, a bacteria or a pathogen.

In a further embodiment, the present disclosure includes a method of determining the presence or absence of an analyte comprising:
a) providing AuNPs, or aggregates of AuNPs, of the present disclosure on a paper substrate;
b) providing a biological, medical or environmental sample comprising the analyte on the paper substrate; and
c) determining the presence or absence of the analyte.
   In a subsequent embodiment, the presence or absence of the analyte in the sample is qualitatively determined by a color change on the paper substrate. The color change being the result of aggregation of the particles, or dissociation of aggregated particles, caused by interaction with the analyte. In a further embodiment, the analyte is a protein, an enzyme, nucleic acid, a small molecule, a metal ion, a bacteria or a pathogen.

In another embodiment, the AuNPs of the present disclosure are useful for the labeling or imaging a target compound in biological or medical samples. Accordingly, the present disclosure also includes a method for the labeling or imaging of a target compound in biological or medical samples comprising:
a) providing a solution of AuNPs, or aggregates of AuNPs, according to the present disclosure;
b) contacting the solution of AuNPs with the biological sample containing a target compound; and
c) optionally imaging the target compound.
   In a subsequent embodiment, the target compound is imaged by microscopy. In another embodiment, the target compound is a protein, an enzyme, nucleic acid, a small molecule, a metal ion, a bacteria or a pathogen.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows a TEM image of AuNP prepared using adenosine 5′-triphosphate (ATP) as the capping ligand in accordance with one embodiment of the present disclosure;

FIGS. 2(a)-(e) show the aggregation of AuNPs according to one embodiment of the present disclosure by the enzymatic conversion of ATP to adenosine;

FIGS. 3(a)-(c) show the aggregation of AuNPs according to one embodiment of the present disclosure by the enzymatic conversion of dNTPs to single stranded DNA;

FIGS. 4(a)-(c) show the aggregation of aptamer-modified AuNPs according to one embodiment of the present disclosure in the presence of a target compound which binds with the aptamer;

FIGS. 5(a)-(b) show a colorimetric AuNP/paper bioassay for protein/enzyme detection according to one embodiment of the present disclosure;

FIG. 6 show colorimetric AuNP/paper bioassays with alcohol coated paper according to one embodiment of the present disclosure;

FIGS. 7(a) and (b) show a colorimetric AuNP/paper bioassay for small molecule detection according to one embodiment of the present disclosure;

FIG. 8 shows a colorimetric AuNP/paper bioassay for nucleic acid detection according to one embodiment of the present disclosure;

FIGS. 9(a)-(c) show a colorimetric AuNP/paper bioassay for small molecule detection using paper chromatography according to one embodiment of the present disclosure;

FIG. 10 shows a colorimetric AuNP/paper bioassays for the detection of metal ions according to one embodiment of the present disclosure;

FIGS. 11(a)-(b) show colorimetric AuNP/paper bioassays for the detection of bacteria according to one embodiment of the present disclosure;

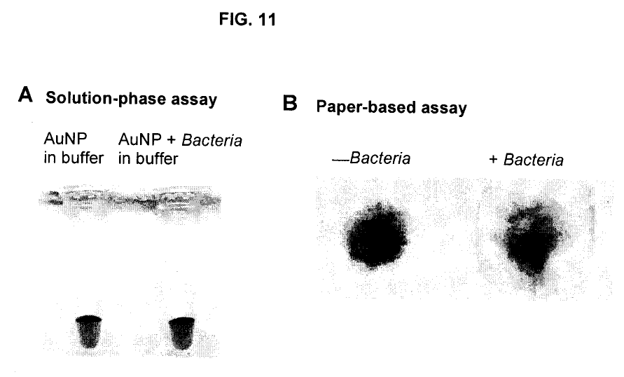

FIG. 12 shows a surface modified AuNP/paper assay according to one embodiment of the present disclosure.

### DETAILED DESCRIPTION

### (I) Definitions

The term “AuNPs” or “AuNPs” refers to gold particles that are generally spherical in shape and which have typically diameters of about 1-100 nm in diameter.

The term “capping ligand” refers to a moiety that has a binding domain and a charged domain wherein the binding domain binds AuNP and the charged (negative or positive) domain protects AuNP from aggregation.

The term “functionalized” means that an accessible surface of the AuNPs comprises additional chemical groupings that, for example, allow attachment of other chemical entities, that provide an altered reactivity for the nanoparticle or that contain recognition or targeting molecules to allow binding or reaction with a target species or molecule of interest.

The term “recognition molecules or entities” or “targeting molecules or entities” refer to chemical groups that will specifically bind to or interact with another molecule or species of interest. These types of molecules and groupings are well known in the art and include, for example, antibodies, aptamers, streptavidin/avidin, etc.

The term “nucleotide” refers to a chemical compound that consists of a heterocyclic base (e.g. adenine, guanine, cytosine, uracil and thymine), a sugar (pentose (five-carbon sugar), deoxyribose or ribose), and one or more phosphate groups. “Oligonucleotides” are short sequences of nucleotides (typically twenty or fewer).

By “water-miscible” and “stable” it is meant that the AuNPs can be well-dispersed in water or buffers with a suitable concentration without any flocculation in a long period of time (e.g. a few months).

The term “monodisperse” refers to the uniformity in size of all AuNPs. By “monodispersed nanoparticles” it is meant the nanoparticles are substantially homogeneous in size. By “substantially homogeneous”, it is meant that the particles vary in size by about ±20%, suitably ±10%.

The term “aggregation” as used herein refers to the association of colloidal particles, in particular, AuNPs. “Inter-particle crosslinking aggregation” refers to the aggregation caused by the inter-particle bridging by crosslinkers whereas “noncrosslinking aggregation” refers to the aggregation process induced by the loss (or screen) of surface charges.

“Enzymatically” refers to chemical reactions catalyzed by enzymes.

DNA (or RNA) “aptamers” refer to the DNA (or RNA) molecules that can specifically bind to their target molecules such as proteins, DNA, small molecules, and metal ions, etc.

DNA (or RNA) enzymes are DNA (or RNA) molecules that are capable of catalyzing chemical reactions.

### (II) AuNP Stabilized with Capping Ligands and Methods for Making the Same

Included within the present disclosure is a water-miscible AuNP, wherein the AuNP is stabilized with at least one capping ligand, the capping ligand having a AuNP binding domain and a charged domain. In an embodiment of the disclosure, the nanoparticle has a diameter of about 1 nm to about 100 nm. In a further embodiment, the diameter of the AuNP is about 1 nm to about 10 nm. In a subsequent embodiment, the diameter of the AuNP is about 2 nm to about 5 nm.

In a further embodiment, the AuNPs of the present disclosure possess an extremely high stability toward salt-induced aggregation. In an embodiment, ATP-capped AuNP was found to be highly stable at high salt concentrations as evidenced by the fact that there was no color change or significant UV-Vis spectral shift when salt concentrations were increased up to at least 1 M NaCl at pH 7.4 for at least a few hours (e.g. 2 h). Without being bound by theory, the extremely high stability of ATP-capped AuNP is most likely due to the fact that the charged phosphate groups prevent particle aggregation via the electrostatic repulsion. These stable AuNPs are therefore “ready-to-use” for biological purpose (e.g. biolabeling and bioimaging) without further surface modifications or ligand exchange reactions.

In an embodiment of the disclosure, the AuNPs are prepared using stabilizing capping ligands having a binding domain and a charged domain. It will be understood by those skilled in the art that the binding domain stabilizes the surface of the AuNP, while the charged domain protects the nanoparticle from the aqueous environment and therefore inhibits aggregation. In an embodiment, the capping ligand is a nucleotide, a deoxynucleotide, a functionalized nucleotide, a nucleoside, an oligonucleotide, a functionalized oligonucleotide, a nucleic acid polymer, a thiol or an amine. In a further embodiment, the nucleotide is adenosine 5′-triphosphate (ATP), adenosine 5′-diphosphate (ADP), adenosine 5′-monophosphate (AMP), guanosine 5′-triphosphate (GTP), cytidine 5′-triphosphate (CTP), thymidine 5′-triphosphate (TTP), inosine 5′-triphosphate or uracil 5′-triphosphate. In another embodiment, the nucleotide is adenosine 5′-triphosphate. One skilled in the art can readily appreciate that other molecules which contain AuNP-binding domain (e.g. nucleobases) and charged domain (e.g. phosphate) can behave analogously to nucleotides.

In another embodiment of the disclosure, the oligonucleotide is a DNA or RNA oligonucleotide. In a further embodiment, the DNA or RNA oligonucleotide is an aptamer that can bind to a target. In another embodiment, the target is a protein, an enzyme, nucleic acid, a small molecule, a metal ion, a bacteria or a pathogen.

In an embodiment of the disclosure, the capping ligand is further functionalized. In another embodiment, the capping ligand is chemically functionalized or enzymatically functionalized.

In another embodiment of the disclosure, when the capping ligand of the AuNP is displaced, the AuNPs form aggregates.

In another embodiment, using different nucleotides (i.e. GTP, CTP and TTP) or adenosine bearing different number of phosphate groups (i.e. ADP, AMP and adenosine) as capping ligands results in AuNPs having different morphologies and size. It was determined that GTP, CTP and TTP also yield significantly smaller AuNPs with narrower monodispersity when compared with AuNPs prepared in the absence of ligands. Based on TEM studies, the efficiency of nucleotides in controlling AuNP size and monodispersity in the present disclosure follows the sequence: ATP>CTP>GTP>TTP, indicating that these nucleotides have different affinities to AuNP. Compared to the other three nucleotides, TTP is the least effective ligand to control AuNP growth, as shown by the formation of relatively larger and more polydisperse AuNP when TTP was used as a capping ligand. It was also confirmed that TTP has a much lower binding affinity to AuNP surface than ATP, GTP and CTP. In addition, ATP- (or GTP-, CTP-) capped AuNPs are stable at >1 M NaCl whereas TTP-capped AuNPs aggregate at about 250 mM NaCl.

In another embodiment, decreasing the number of phosphate groups in the nucleotide used as capping ligand for the formation of AuNPs (i.e., from ATP to ADP, AMP and adenosine), resulted in more aggregated or fused AuNPs, as confirmed by TEM images. This was further confirmed by the UV-V is spectra, which showed that the surface plasmon band becomes broader as the phosphate group number in the ligands decreases, and is an indication of AuNP aggregation.

In further embodiments of the disclosure there is included a method for the production of monodisperse water-soluble AuNPs. In an embodiment, the method comprises reacting a solution of a gold salt in a suitable solvent with a stabilizing capping ligand and a reducing agent, wherein the capping ligand has a binding domain and a charged domain.

In another embodiment, the gold salt is HAuCl₄. In a further embodiment, the HAuCl₄ is present in the suitable solvent in an amount of about 100 μM to about 100 mM. In another embodiment, the HAuCl₄ is present in the suitable solvent in an amount of about 100 μM to about 10 mM. In a further embodiment, the solvent is water.

In another embodiment of the present disclosure, the stabilizing capping ligand is a nucleotide. In another embodiment, the nucleotide is adenosine 5′-triphosphate (ATP), adenosine 5′-diphosphate (ADP), adenosine 5′-monophosphate (AMP), adenosine, guanosine 5′-triphosphate (GTP), cytidine 5′-triphosphate (CTP), thymidine 5′-triphosphate (GTP), inosine 5′-triphosphate or uracil 5′-triphosphate. In a further embodiment, the nucleotide is adenosine 5′-triphosphate (ATP).

In an embodiment of the disclosure, the stabilizing capping ligand is present in an amount of about 100 μM to about 100 mM. In a further embodiment, the stabilizing capping ligand is present in an amount of about 100 μM to about 10 mM.

In another embodiment of the present disclosure, the reducing agent is sodium borohydride (NaBH₄). In a further embodiment, the sodium borohydride is present in an amount of about 100 μM to about 500 mM. In another embodiment, the sodium borohydride is present in an amount of about 100 μM to about 100 mM.

In an embodiment of the present disclosure, the reaction is performed at a temperature of about 10° C. to about 50° C. In another embodiment, the reaction is performed at about room temperature. In a subsequent embodiment, the reaction is performed for a period of about 1 hour to about 5 hours. In a further embodiment, the reaction is performed for about 3 hours.

In another embodiment of the present disclosure, the molar ratio of the gold salt to the capping ligand ([gold salt]:[capping ligand]) is about 0.1 to about 10. In another embodiment, the molar ratio of the reducing agent to the gold salt ([reducing agent]:[gold salt]) is about 15 to about 20. In another embodiment, the size of the stable AuNPs is controlled by the molar ratios of the gold salt, the capping ligand and the reducing agent.

In an embodiment of the disclosure, it has been determined that the size of AuNP during formation is controlled in the range of 2 nm-5 nm. A person skilled in the art may adjust the type of capping ligand used, or the molar ratio of the gold salt, nucleotides and reducing agents (e.g. NaBH₄). In one embodiment of the disclosure, higher [nucleotides]/[HAuCl₄] or higher [NaBH₄]/[HAuCl₄] results in AuNPs with smaller sizes.

In an embodiment of the disclosure, as the molar ratio of the gold salt to the capping ligand decreases, the size of the AuNP decreases (e.g. 2.73±0.8 nm for [capping ligand]:[gold salt]=10:1). This is further confirmed by UV-V is adsorption spectra which showed that the surface plasmon band became less distinct as more ATP was added, indicating that AuNPs with smaller sizes were formed. The AuNPs prepared in the presence of more ATP also showed a better control of size dispersity.

In another embodiment of the disclosure, when the molar ratio of the reducing agent to the gold salt was varied, the results indicated that the molar ratio required to produce monodisperse spherical AuNP generally ranged from 15 to 20. When less or more of the reducing agent was added, poorly size controlled (e.g. fused or elongated) particles were observed in TEM. Without being bound by theory, insufficient reducing agent results in a slower crystal growth process leading to difficulties in controlling the crystal size and morphology. Surprisingly, an excess of reducing agents also yielded poorly controlled AuNPs. Excess reducing agent resulted in such a rapid crystal growth process that the capping ligand may no longer effectively stabilize the growing particles.

In another embodiment of the present disclosure, the reaction is performed under environmentally friendly conditions (e.g. the use of nontoxic capping ligands, room temperature and water as solvent) in one step. The particle sizes are reasonably monodisperse and can be easily tuned in 2 nm-5 nm by adjusting reaction conditions.

In another embodiment of the disclosure, the resulting nucleotide-capped AuNPs are optionally surface modified. Capping ligands (e.g. thiols) with higher binding affinity to AuNP can displace the bound nucleotides so that a wide variety of functionalities can be introduced. Surface functionalization can also be conducted directly onto the nucleotide-capped AuNPs through chemical approaches (e.g. EDC coupling to phosphate) or enzymatic approaches (e.g. phosphate targeting proteins).

In an embodiment of the disclosure, the concept of non-crosslinking AuNP aggregation is introduced to construct simple and rapid colorimetric biosensors. The SPR property makes AuNPs suitable reporters for colorimetric biosensors based on the principle that the well-dispersed AuNP appears red in color whereas the aggregated AuNPs have a blue (or purple) color. Most of the aggregation mechanism in previously reported AuNP-based biosensors relied on inter-particle crosslinking (e.g. DNA hybridization, antibody-antigen interaction or peptides). For example, see Elghanian, R. et al, Science, 277, 1078 (1997); Guarise, C. et al. Proc. Natl. Acad. Sci. USA. 103, 3978 (2006); Choi, Y. et al. Angew. Chem. Int. Ed 46, 707 (2007). In the present disclosure, AuNP-based colorimetric biosensors are developed based on non-crosslinking AuNP aggregation. Colloidal stability can be adjusted by modifying surface charges that affect electrostatic stabilization, and that aggregation can be induced due to the loss (or screening) of surface charges. The approaches to reduce surface charges include, for example, using non-charged molecules to displace charged motifs on the AuNP surface or by removing charged molecules from the surface.

AuNPs with controlled sizes, shapes, and monodispersity can be prepared in a well-defined fashion according to the present disclosure. Note that the physical properties (particularly the colors) are dependent on the AuNP size, shape and structures. One would therefore expect the present disclosure, may be extended to AuNPs with different sizes, shapes (e.g. gold nanorods, nanowires, etc.) and other nanostructures (e.g. gold nanofoams, nanoshells, nanocages, etc.). Moreover, the optical properties of AuNPs are also dependent on AuNP surface modifications which can be readily introduced by straightforward surface chemistry (e.g. Au—S interaction). Furthermore, the introduction of other types of nano-scaled materials, including silver nanoparticles, carbon nanotubes, silicon nanowires, quantum dots, magnetic nanoparticles, SiO₂ (TiO₂) nanoparticles, among others, could further expand the functions of the present paper-based bioassays. Therefore, as will become apparent to those skilled in the art, a large variety of assays using nano-scaled materials as signal transducers can be constructed simply by conducting various changes and modifications within the spirit and scope of the present invention.

In the colorimetric AuNP/paper bioassays, AuNPs can be used as prepared without any further surface modifications (in the case of AuNPs prepared by citrate reduction, for instance) or with further surface modification using, for instance, biomolecule receptors including DNA, proteins, among others. For instance, various forms of DNA including aptamers are widely used as receptors (or biorecognition motifs) in biosensors. DNA-modified AuNPs can be prepared via thiol-modified DNA and AuNPs. Protein (including antibodies and enzymes) can also be used as receptors on AuNPs and can be conjugated through their cysteine tags and other chemical interactions. Other biomolecules such as nucleotides, peptides, amino acids, sugars can also be applied and conjugated based on the standard bioconjugation techniques known in the art.

### AuNP Formation Mechanism

Without being bound by theory, the binding of nucleotide on an AuNP surface during the crystal growth process results in highly negatively charged nanoparticles due to the presence of phosphate groups. The negatively charged phosphate groups thus play significant roles in protecting AuNPs against aggregation during the crystal growth process and, therefore, in controlling the size and morphology of AuNPs.

Any process that leads to loss of charged groups from the surface of AuNPs leads to their aggregation with an immediate and obvious colour change (see Scheme 1). Thus, any chemistry, or biochemistry (e.g. enzymatic transformation) that leads to changes in surface charge, by consumption/conversion of the surface bound materials, will manifest itself in a color change. Some specific non-limiting examples are provided.

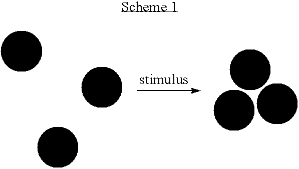

In an embodiment of the disclosure, if a nucleotide is the capping ligand, enzymatic dephosphorylation may lead to aggregation, as seen in Scheme 2.

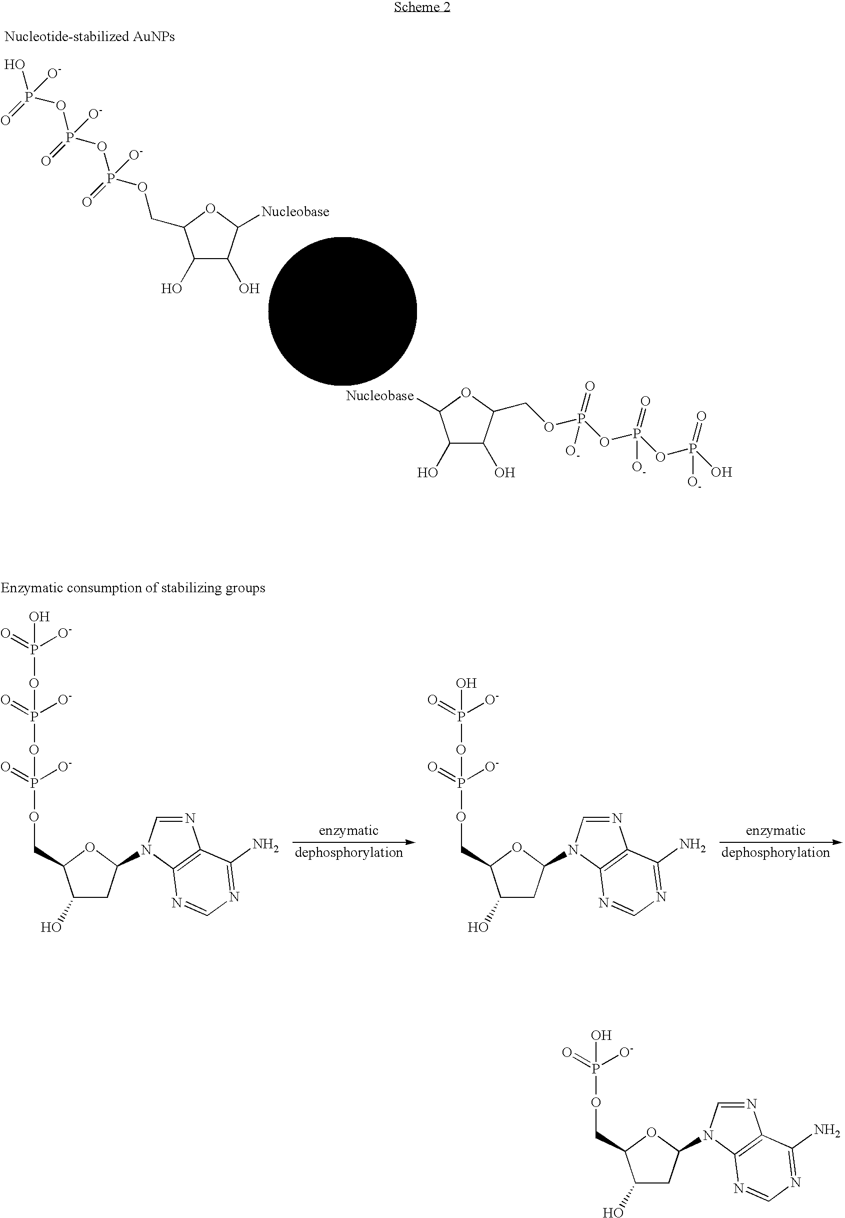

In another embodiment, if a nucleoside is the capping ligand, DNA polymerization may lead to aggregation of the AuNPs as seen in Scheme 3.

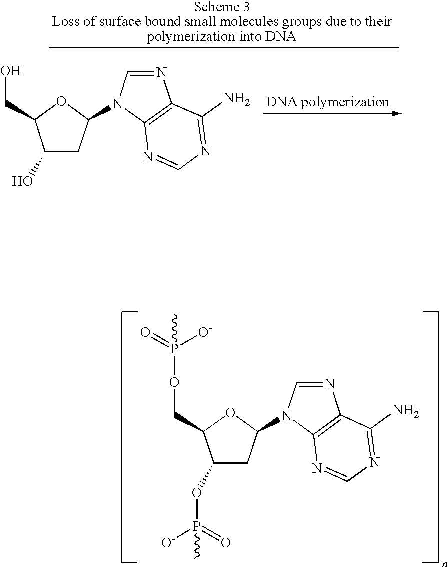

In another embodiment, when the capping ligand is an oligonucleotide hybridized to an aptamer, the addition of a target for the aptamer may also lead to aggregation as seen in Scheme 4.

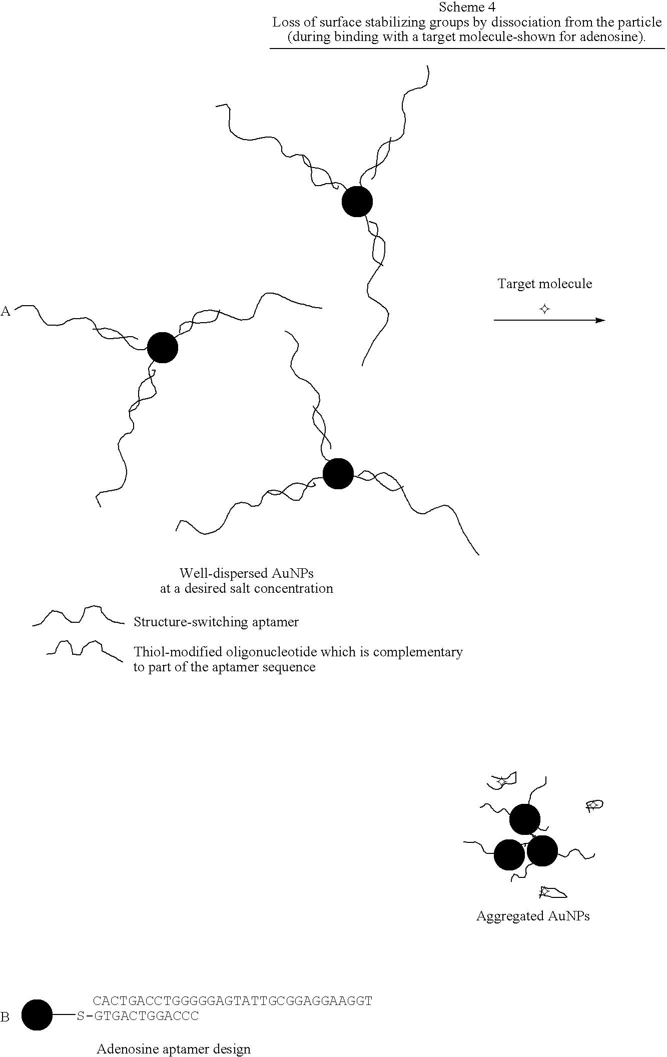

In a further embodiment of the application, when an oligonucleotide is the capping ligand, the addition of an enzyme able to cleave the nucleic acid may lead to aggregation of the AuNPs, as seen in Scheme 5.

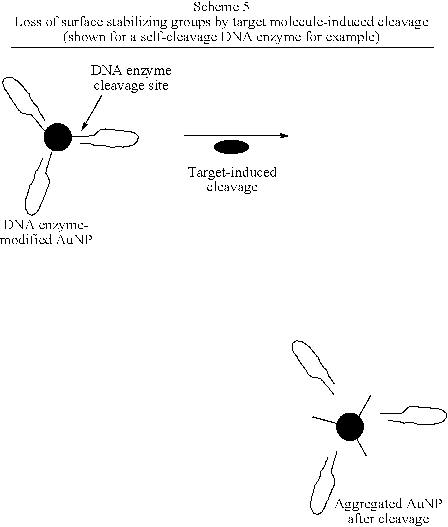

In another embodiment, when the capping ligand is an oligonucleotide, the addition of a target which is able to conformational alter the shape of the nucleic acid may also lead to aggregation, as seen in Scheme 6.

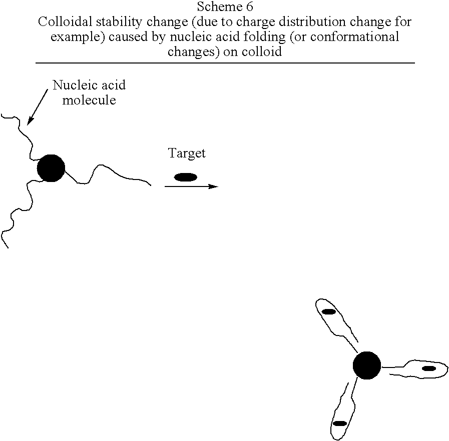

### (III) Uses of AuNPs and Assays Containing AuNPs

The present disclosure includes water-soluble, highly stable, small AuNPs (diameter 2 nm-5 nm). AuNPs with small size (typically <5 nm) are proven to be suitable materials for use in certain nanodevices (for example, see Andres, R. P, et al. Science, 272, 1323 (1996)), biosensors (for example, see Dubertret. B, et al. Nat. Biotechnol. 19, 365 (2001)) and biolabels (for example, see Birrell, G. B. et al. Histochem. Cytochem, 35, 843 (1987)) due to their small size and unique physical properties.

The present disclosure also includes the construction of AuNP biosensors based on a unique non-crosslinking AuNP aggregation mechanism induced by the loss of surface charges (surface stabilization ligands). Nanoparticle aggregation can be induced due to the loss (or screening) of surface charges, which decreases electrostatic stabilization. As such, AuNP biosensors can be used to detect biological analytes that can modify the surface charges (for example, using non-charged molecules to displace charged molecules on AuNP surface or switching the charged DNA molecules off the surface in the presence of target molecules of interest).

In an embodiment of the disclosure, the nucleotide-capped AuNPs and biosensors based on non-crosslinking AuNP aggregation is useful in many applications. All uses of these applications and uses are included within the scope of the present disclosure.

In another embodiment of the disclosure, the monodisperse small AuNPs are used for nanodevices including nano-electronics and photonics. The stable AuNPs can be directly used under physiological conditions for biological labeling and imaging. For example, nucleotide-capped AuNP can be used to specifically label phosphate-targeting proteins in vitro or vivo. The AuNP-labeled biomolecules are visualized using electron microscopy. The AuNPs are also used as biological indicators for biodetection of a large variety of species. For example, surface functionalization of these AuNPs with antibody or DNA aptamer that can specifically bind to the receptor on bacteria membrane allow pathogenic bacteria to be labeled and then detected. Moreover, these AuNPs can be use to detect proteins or small metabolites that are responsible for certain diseases such as cancer. This observation is based on the SPR properties of AuNPs or the fact that AuNP can quench fluorescence (for example, upon binding target molecules, the fluorophore-labeled DNA molecules undergo structure switches that moves the fluorophore away from AuNP quencher and thus fluorescence signal can be observed. Therefore, colorimetric assays or fluorescent assays can be developed using these AuNPs. The AuNPs are also advantageously printable onto various substrates including paper (for example, a filter, trap or support).

The simple and rapid colorimetric assay based on non-crosslinking AuNP aggregation can be used for detection a large variety of analytes such as proteins, enzymes, nucleic acid, small molecules, metal ions, and bacteria. The noncrosslinking AuNP aggregation is induced by the loss of charged motifs on AuNPs. For example, the assay can be adapted to detect enzymes that consume stabilizing groups on AuNPs. The dissociation of charged DNA aptamers (or DNA enzymes) on AuNPs upon binding of target molecules can be used to detect proteins and small metabolites. Furthermore, AuNP stability is changed by target molecule-induced nucleic acid folding (or conformational change) on AuNP surface even though there is no charge loss during the folding process. This AuNP aggregation (or stabilization) phenomenon induced by nucleic acid folding can be used to detect responsible target molecules and to investigate the nature of nucleic acid folding.

Accordingly, the present disclosure includes a method of monitoring or detecting a substance or process that induces aggregation of AuNPs, or dissociation of AuNP aggregates, comprising contacting the process or substance with the AuNPs of the present disclosure and observing or detecting a color change due to the aggregation or dissociation of the particles, wherein a color change is indicative of the substance or process.

### Enzyme Assays

In an embodiment of the disclosure, a typical enzyme sensing assay solution containing ATP, MgCl₂, Tris-HCl (pH 7.5), and CIAP (alkaline phosphatase), is monitored by removing aliquots from the reaction mixture and mixing them with an AuNP solution. The color of the solution changes progressively from red to blue, which indicates the aggregation of AuNP during the conversion of ATP to adenosine.

In another embodiment of the disclosure, colorimetric AuNP biosensors based on non-crosslinking aggregation are used to monitor enzymatic reactions (or sensing enzymes). If the reactant and product of an enzymatic reaction differently affect the AuNP stability by changing their electrophoretic properties, such a reaction can be monitored colorimetrically using AuNPs. Specific examples include, but are not limited to, the enzymatic reactions concerning nucleoside triphosphates as substrates, specifically nucleotide dephosphorylation by alkaline phosphatase. For example, the present disclosure includes an AuNP-based assay to monitor an enzymatic reaction where ATP is converted into adenosine by calf intestine alkaline phosphatase (CIAP). Given the fact that nucleobases can bind to citrate-capped AuNPs with the displacement of weakly bound citrate ions via metal-ligand interactions, the adsorption of highly charged nucleotides (ATP) or uncharged nucleosides (adenosine) further stabilize AuNPs or cause their aggregation, respectively, due to the gain or loss of surface charges. Taking advantage of this, the color change of AuNPs after mixing with enzymatic reaction solution indicates how much ATP (reactant) has been converted into adenosine (product) or how far of the reaction has proceeded. UV-Visible adsorption spectroscopy is used to quantify the color change, and therefore the enzyme activities can also be quantified.

In other embodiments of the disclosure, the AuNPs are adapted to enzymatic reactions where the reactants and products impact differently on the AuNP stability by changing their electrophoretic properties. The substrates could be oligonucleotides, amino acids, and peptides, etc.

### Aptamer Assays

In another embodiment of the disclosure, simple and rapid colorimetric assays based on non-crosslinking AuNP aggregation and structure-switching DNA aptamers have been developed to detect small molecules. The term “DNA (or RNA) aptamers” refer to the DNA (or RNA) molecules that can specifically bind to their target molecules such as proteins, DNA, small molecules, and metal ions, etc. A structure-switching aptamer is a DNA or RNA aptamer that undergoes a structure switch from aptamer/complementary DNA duplex to aptamer/target complex because the aptamer preferentially binds to its target molecule. For example, see Nutiu, R, et al. J. Am. Chem. Soc. 125, 4771 (2003). By hybridizing a structure-switching DNA aptamer (e.g. adenosine aptamer) with a short oligonucleotide attached on AuNP surface, the AuNPs are highly stable at a relatively high salt concentration (30 mM MgCl₂) due to both electrostatic and steric stabilization. In the presence of target molecules, the DNA aptamers switch off from AuNP surfaces, leading to the aggregation of instable AuNPs. The red-to-purple color change indicates the presence of target molecules.

In another embodiment of the present disclosure, a simple and rapid colorimetric assay that exploits structure-switching DNA aptamers and non-crosslinking AuNP aggregation is exploited. Conceptually, as shown in Scheme 4, the structure-switching DNA aptamer is first hybridized with a short complementary oligonucleotide that is attached on AuNPs. At relatively high salt concentrations, the aptamer dissociates from the AuNPs upon binding of target molecules, which causes the AuNP aggregation and thus a red-to-purple color change.

Since DNA (or RNA) aptamers can be obtained from in vitro selection and therefore, in principle, aptamers for any target of interest can be available, this assay can be easily applied to detect other target molecules such as proteins, DNA, small molecules, and metal ions, etc.

Besides structure-switching aptamers, other functional DNA (or RNA) molecules such as DNA (or RNA) enzymes, riboswitches, etc. can also be able incorporated with the non-crosslinking AuNP aggregation mechanism to construct biosensors.

In another embodiment of the disclosure, a colorimetric assay using non-crosslinking AuNP aggregation and structure-switching DNA aptamer was developed to detect small molecules such as adenosine. A thiol-modified short oligonucleotide that is complementary with part of the aptamer sequence is first attached with 13 nm AuNPs via Au—S chemistry. An optional step using a 6-mercapto-1-hexanol (MCH) ligand exchange reaction is used to remove the nonspecifically adsorbed and some of the thiol-tethered oligonucleotides. The loss of oligonucleotides on AuNP surface leads to a decrease of AuNP stability. Therefore, the AuNP stability can be tuned in a wide range simply by adjusting the MCH concentration or treatment time. Moreover, MCH treatment increases the hybridization efficiency in the subsequence aptamer hybridization step because of the reduced steric hindrance after the dilution of oligonucleotide concentration on AuNP surface. After MCH treatment, the structure-switching DNA aptamer is attached onto AuNPs through hybridization with short oligonucleotides.

### Colorimetric AuNP/Paper Assays

Paper is a web formed from cellulose fibers with the structure that is highly controllable. The surface nature of the material is readily modified to be hydrophobic (e.g. for printing, by use of sizing agents) or hydrophilic (by, for example, polyvinyl alcohol), stronger (by addition of wet and dry strength polymers), brighter or colored by the addition of pigments, and made more opaque and stronger by the addition of mineral fillers (e.g. clay, silica).

Paper-based bioassays using AuNP probes are bioassays that use paper as substrate (or support) on (or in) which AuNPs (often modified with biomolecule receptors such as DNA) are used as colorimetric reporters.

Paper chromatography is an analytical technique for separating and identifying mixtures. Traditionally, a small concentrated spot of sample solution is applied to a strip of paper. The base of the paper is then inserted in a suitable solvent (developing solution) that moves up the paper by capillary action, which separates the compound mixtures in the sample taking advantage of the differential adsorption of the solute components. A similar concept has been applied in the present disclosure where AuNP probes are spotted on paper. The target analyte can be co-spotted with AuNP probes on paper or can be included in the developing buffer. The separation and detection of the target analytes are achieved by the biorecognition of target and AuNP probes, which modulates the AuNP optical properties and generates a colorimetric detectable signal.

In an embodiment of the present disclosure, the AuNPs, regardless of their format (including well-dispersed, aggregated, surface modified or unmodified) can be spotted onto paper, and therefore used as paper assays. AuNPs show similar (or enhanced) colors on papers to those in solution. A wide variety of papers can be used in the present disclosure, ranging from pure cellulose to polymer modified, mineral filled materials that are modified with surface coatings. The structure of paper is highly controllable, and the surface nature of the paper material is readily modified to be more hydrophobic (or hydrophilic), stronger or more flexible (by addition of wet and polymers), brighter and made more opaque and stronger by the addition of mineral fillers (e.g. clay, silica).

In an embodiment of the disclosure, after preparation of a paper assay, the AuNP probe-coated papers are dried briefly (e.g. ˜15 min in air), and then immediately used for a bioassay. In some embodiments, some moisture is required to maintain biomolecule activity. Alternatively, the AuNP probe-coated papers can be completely dried, and they can still be used for biodetection. When the AuNP paper assays are dried completely, the paper assay is dried in an oven at 90° C. for 1 h. Completely dry paper assays still show the expected color and color changes that are used for biodetection applications. This is highly beneficial from the practical application standpoint given that dried paper would be ideal for portable and easy-to-use (and carry) purposes.

In another embodiment of the disclosure, the use of colorimetric AuNP/paper bioassays can be conducted in a variety of ways. In some cases, one can directly drop sample solution onto paper where AuNP probes are spotted. The color change indicates the presence of target analytes. Alternatively, AuNP probe coated papers are dipped in a developing buffer that contains target analytes. The target analytes migrate with the buffer along paper and eventually react with AuNP probes which generate a color signal on paper.

It will be understood by those skilled in the art that the preparation of sample solutions depends on the specific target and assay used. In many cases, no pre-separation is required. Such cases include, but are not limited to, the use of colorimetric AuNP/paper assays for the detection of toxic metal ions (e.g. Hg²⁺) and pathogens in drinking water, and many other clinical tests such as a pregnancy test. In some other cases, where target analytes are in a mixture that interfere with assay performance, pre-separation steps are required. Sample clean up in these cases can be readily achieved using traditional methods known in the art including high performance liquid chromatography.

Colorimetric AuNP/paper bioassays have many attractive features: (1) paper provides an inexpensive platform for economical, low-volume, portable, disposable, and easy-to-use bioassays; (2) the assays are very sensitive due to the extremely high extinction coefficient, which is normally ˜1000 higher than that of common dyes; (3) the assays are selective to the target analyte of interest due to the use of specific biomolecule receptors such as DNA, DNA aptamers and antibodies, etc.; (4) the assay is very rapid: the color signal in a typical assay is observed in a few seconds to a few minutes; (5) the colors can be observed by naked eye and no sophisticated instruments are required; (6) the assay is highly generic because AuNP aggregation can be initiated by a wide variety of structurally different target analytes including, for example, using DNA aptamers and antibodies using the principles described above; and (7) the assays are highly versatile because the AuNP aggregation is generally not sensitive to the nature of the paper substrate or the presence of surface coatings.

The following non-limiting examples are illustrative of the present disclosure:

### EXAMPLES

### Materials and Methods

HAuCl₄, ATP, TTP, CTP, GTP, ADP, AMP, adenosine, trisodium citrate, MCH, guanosine, cytosine, inosine, ethyl acetate, Pb(OAc)₂, HgCl₂, DNase I, polyvinyl alcohol and NaBH₄ were purchased from Sigma. Calf intestine alkaline phosphatase (CIAP), phi29 DNA polymerase, and dNTPs (dATP, dCTP, dTTP, and dGTP) were purchased from MBI Fermentas. γ-³²P ATP was obtained from Amersham Biosciences. Thiol-modified DNA was obtained from Keck Biotechnology Resource Laboratory, Yale University. Unmodified DNA was purchased from Central Facility, McMaster University. H₂O was doubly deionized and autoclaved before use. 13 nm AuNPs (AuNP) were prepared according to the classic citric reduction method and the final concentration was estimated to be about 14 nM. Filter paper (Whatman #1) was used as received.

The (HR) TEM sample was prepared by dropping AuNP solution (4 μL) onto a carbon-coated copper grid. After 1 min, the solution was wicked from the edge of the grid with a piece of filter paper. The TEM and HRTEM images were measured with a JEOL 1200 EX and Philips CM12 transmission electron microscope, respectively. UV-Vis adsorption spectra were measured using a Cary 100 UV-Vis spectrophotometer. For XPS and XRD experiments, samples were prepared as follows: AuNPs were precipitated by centrifugation at 45,000 rpm for 30 min. The pellet can be easily redispersed in ddH₂O (1 mL). The solution was centrifuged again and redispersed in ddH₂O for XPS experiments or dried at room temperature under vacuum for 3 days for XRD experiments. For XPS experiment, a few drops of AuNP solutions were put on a glass substrate and dried in air. XPS experiments were conducted on a Leybold Max 200, magnesium anode nonmonochromatic source spectrometer.

Peak positions were internally referenced to the C1s peak at 285.0 eV. XRD was performed using a X-ray diffractometer with Cu Kα radiation (wavelength λ=0.154 nm) operated at 40 kV and 40 mA. Electrophoretic mobilities of nanoparticles were measured at room temperature using a ZetaPlus (Brookkhaven Instruments Corporation). The reported values were based on 10 measurements with 15 cycles for each sample.

### Preparation of Bacterial Targets

Frozen glycerol stocks of bacterial strains Escherichia coli (E. coli) and Bacillus subtilis (B. subtilis) were inoculated on Luria-Bertani (Sigma LB L3022) agar plates overnight at 37° C. Single colonies were picked from incubated plates and inoculated in 3 mL Luria-Bertani broth (14 mL Falcon polystyrene round bottom culture tubes 350275) and grown to an OD₆₀₀ of ˜0.8 at 37° C. at 260 r.p.m on a shaker-incuabtor (New Brunswick Scientific C24 incubator-shaker). The bacterial suspensions were then extracted by centrifugation at 5000×g for 5 minutes. The bacterial pellet was then resuspended in double-deionized MilliQ water. This was repeated twice to minimize residual contaminants from the growth medium. A final suspension in double-deionized MiliQ water was prepared to a concentration of 10⁷ CFU/mL (Colony Forming Units). Aliquots from this suspension were subsequently used in the assays.

### Example 1

### Preparation of Citrate-Capped AuNPs

Trisodium citrate (25 mL, 38.8 mM) was added to a boiling solution of HAuCl₄ (250 mL, 1 mM). Within several minutes, the color of the solution changed from pale yellow to deep red. The mixture was allowed to heat under reflux for another 30 min to ensure complete reduction, before it was slowly cooled to room temperature, and stored at 4° C. before use. The concentration of these AuNPs was 13.4 nM as determined by UV-Visible spectroscopy. The diameter of the AuNPs was 13 nm.

### Example 2

### General Procedure for the Preparation of Nucleotide-Capped AuNPs

A solution of HAuCl₄ (10 mM, 60 μL) in water and ATP (10 mM, 60 μL) were added into 2.75 mL ddH₂O in a 4 mL glass vial, and the mixture was incubated at room temperature for 30 min. Freshly prepared NaBH₄ solution (100 mM, 100 μL) was then quickly added, and the vial was vigorously shaken for 10 s. The reaction was left at room temperature for at least 3 h to ensure the completion of AuNP growth.

### Discussion

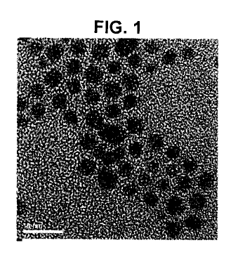

In a typical AuNP synthesis process, an orange color appeared immediately upon the addition of NaBH₄, indicating the formation of AuNP. The color of the solution changed gradually from orange to red over 2 h, after which there was no further color change, indicating the AuNP growth was complete within 2 h. As seen in the TEM image of FIG. 1, the ATP-capped AuNPs, which was prepared with a molar ratio of starting materials of [HAuCl4]:[ATP]:[NaBH4]=1:1:16.5, displayed well-distributed spherical nanocrystals with small sizes and improved dispersity (3.75±1.1 nm). UV-Vis spectroscopy of ATP-capped AuNP showed a small and broadened surface plasmon band around 510 nm, which is characteristic for small spherical AuNPs. HRTEM and XRD results reveal the crystalline nature (face-centered cubic (fcc) structure) of the ATP-capped AuNPs. XPS spectra clearly showed two peaks centered at binding energies of 83.9 and 87.6 eV, which correspond to the Au 4f_{7/2} peak and Au 4f_{5/2} peak, respectively, indicating the formation of the metal AuNP. The binding of ATP to AuNP is demonstrated by the presence of N1s and P2p at binding energies of 400.1 and 134.2 eV, respectively.

### Example 3

### Non-Crosslinking AuNP Aggregation Assay for Sensing CIAP

A series of 20 μL enzymatic reaction solutions were made which contained ATP (200 nmol, 10 mM), reaction buffer (10 mM MgCl₂, 10 mM Tris-HCl (pH 7.5)), and various amounts of CIAP (from 10 units to 0.16 units). At various times, 1 μL of reaction solution was taken and added into 200 μL AuNP (14 nM). UV-Vis spectra are then recorded, and the spectra shift were used to quantify the color change and enzyme activities.

### Discussion

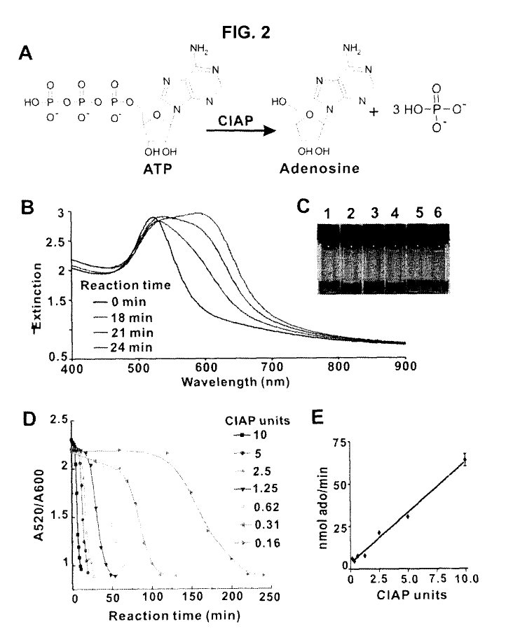

As seen in FIG. 2, (A) shows the enzymatic reaction in which ATP is converted into adenosine by CIAP. (B) shows UV-Vis spectra of AuNP solutions after addition of ATP-CIAP mixture incubated at the indicated time points, while (C) shows photographs of AuNP solutions after addition of the ATP-CIAP mixture incubated for 0, 18, 21 and 24 min (vials 1-4, respectively). Vials 5 and 6 were the AuNP solutions with addition of the mixture (incubated for 24 min) where ATP or CIAP was omitted, respectively. As seen in (C), the reaction mixture in vials 1-4 becomes progressively darker as the AuNPs begin to aggregate as a result of the loss of the capping ligand ATP. (D) is a graph showing A520/A600 vs. reaction time for the indicated CIAP concentrations, while (E) is a graph showing the amount of substrate processed per minute vs. CIAP concentration (units/20 μL reaction solution). As the enzymatic reaction time increased, the color of the solution changed progressively from red to blue, and the UV-Vis adsorption spectra (originally peaked at ˜520 nm) broadened and shifted to longer wavelength. The ratio of absorbance at 520 nm and 600 nm (A520/600) is plotted as a function of reaction time in FIG. 2 (D). The enzyme activity can then be quantified (FIG. 2).

Typically, when 2.5 units CIAP is used, only a few minutes (e.g. 5 min) of reaction time is required in order to see an instant red-to-blue color change of AuNP solution. As CIAP concentration decreases, the reaction time needed to give a quick color change in the subsequent AuNP test assay increases. The typical detection range for CIAP in the current study is −0.16 units to 10 units. It is also important to note that the minimum detectable substrate (ATP) concentration is ˜300 μM. In the control experiments where reactant (ATP) or CIAP were absent, no color change of AuNP solution was observed.

### Example 4

### Non-Crosslinking AuNP Aggregation Assay for Sensing phi29 DNA Polymerase

A 20 μL enzymatic reaction solution contained dNTPs (2 mM dGTP, 3 mM dATP, 0.6 mM dCTP, and 0.7 mM dTTP), phi29 DNA polymerase (40 units), primer (5′-GGCGAAGACAGGTGCTTAGTC, 20 pmol, 1 μM), circular template (5′-TGTCTTCGCCTTCTTGTTTCCTTTCCTTGAAACTTCTTCCTTTCTTTCTTTC GACTAAGCACC, 20 pmol, 1 μM), 1× reaction buffer (33 mM Tris-acetate (pH 7.9 at 37° C.), 10 mM Mg-acetate, 66 mM K-acetate, 1% (v/v) Tween 20). The reaction was performed at 37° C. At certain time intervals, 1 μL of the reaction solution was taken and added to 99 μL NaCl solution (75 mM). This solution was then added to 200 μL AuNP solution (14 nM) and the UV-Vis adsorption spectra were measured 1 min after mixing.

### Discussion

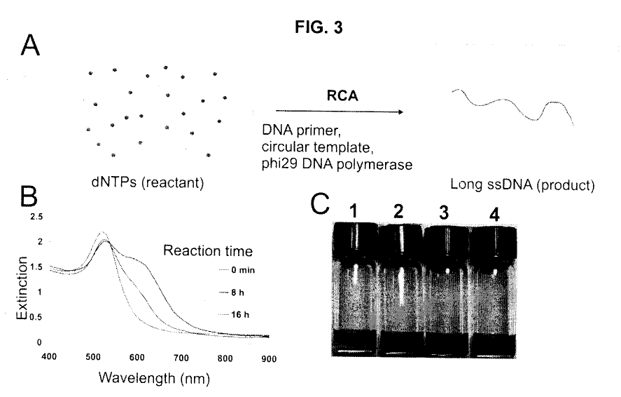

As seen in FIG. 3, (A) shows the DNA polymerization via rolling circle amplification: dNTPs (reactant) are converted into long ssDNAs (product). As seen in (B), a representative UV spectra of AuNP solution after the addition of phi29 enzymatic reaction solutions taken at different reaction time. (C) Photographs of AuNP solutions after the addition of phi29 enzymatic reaction solutions with reaction times at 0 (vial 1), 8 h (vial 2) and 16 h (vial 3), respectively. As seen, the solution in the vials becomes progressively darker as the dNTPs are consumed and are not able to act as a capping ligand for the AuNPs. Vial 4 was the AuNP solutions after the addition of a control enzymatic reaction solution quenched at 90° C. for 10 min right after the addition of phi29 DNA polymerase. The control reaction was conducted for 16 h.

dNTPs bind AuNP more effectively than the long ssDNA product because of the steric hindrance and large secondary structures formed in long ssDNA, and that dNTPs would stabilize AuNP more effectively than the long ssDNA. Since dNTPs and the long ssDNA product both stabilize AuNPs (although to different extents), the assay was performed at a specific salt concentration where dNTP/AuNP is stable whereas long ssDNA/AuNP would aggregate. The AuNP solution mixed with the reaction mixture that either excluded or contained DNA polymerase was red and blue in color, respectively. The red-to-blue color change indicated that AuNP aggregation occurred as dNTPs were made into long ssDNA by phi29 DNA polymerase.

### Example 5

Non-crosslinking AuNP aggregation assay for sensing adenosine Short oligonucleotide-attached AuNPs (AuNP-OD) were prepared using thiol-modified oligonucleotide (5′-CCCAGGTCAGTG-thiol-3′) (280 μL, 6.6 μM) which was mixed with AuNP solution (13 nm in diameter, 600 μL, ˜13 nM). The solution was incubated at room temperature for 45 h. Tris-HCl buffer (10 μL, 1 M, pH 7.4) and NaCl solution (90 μL, 1 M) was added and the mixture was incubated for another 28 h. Tris-HCl buffer (5 μL, 1 M, pH 7.4) and NaCl solution (50 μL, 5 M) were added and the mixture was further incubated for 18 h at room temperature. The solution was then centrifuged at 14 000 rpm for 15 min. The precipitated AuNP-OD was washed twice by 1 mL washing buffer (20 mM Tris-HCl (pH 7) through centrifugation. Finally, the AuNP-OD was redispersed in 600 μL wash buffer.

The as-prepared AuNP-OD solution was then diluted by an equal volume of wash buffer and MCH was then added with a final MCH concentration of 5 μM. The MCH treatment was performed at room temperature for 2 h. The reaction was quenched by three washes with equal volumes of ethyl acetate. The structure-switching aptamer-attached AuNPs (AuNP-OD-APT) was then prepared as follows: adenosine aptamer (5′-CACTGACCTGGGGGAGTATTGCGGAGGAAGGT-3′) (19.8 μL, 55.5 μM) was added to AuNP-OD solution (1 mL, ˜6 nM). The hybridization solution was slowly cooled from 70° C. to room temperature over about 1 h. The solution was then centrifuged and washed once by an equal volume of wash buffer and finally redispersed in 1 mL wash buffer. Various amounts of adenosine (from 10 μM to 2 mM) were then added to initiate detection. An instant red-to-blue color change can be observed when high adenosine concentration (≧500 μM). Using UV-Vis spectroscopy, the detection limit is about 10 μM.

### Discussion

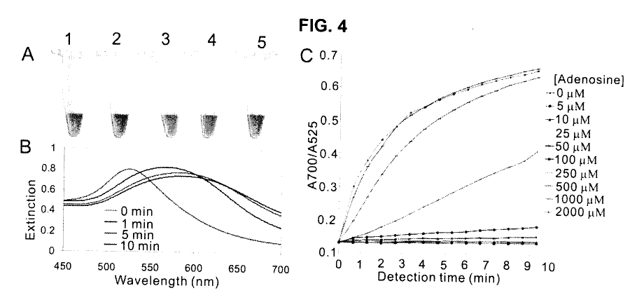

As seen in FIG. 4, (A) shows a series of photographs of AuNP-OD-APT solution in the absence of target (tube 1) and in the presence of 1 mM adenosine (tube 2), inosine (tube 3), guanosine (tube 4) and cytosine (tube 5). Photos were taken 1 min after the addition of concerned nucleosides. (B) shows a representative UV-Vis spectra of AuNP-OD-APT in the present 1 mM adenosine at different detection time, while (C) shows the kinetics of AuNP aggregation in the presence of various amounts of adenosine.

The first step in the present Example is the conjugation of a short oligonucleotide strand, which is complementary to part of the structure-switching aptamer sequence, to AuNPs so that the DNA aptamer is subsequently attached to the AuNP surface through hybridization. These short oligonucleotide-attached AuNPs (referred as AuNP-OD) were prepared via Au—S chemistry. To determine the number of oligonucleotides on each AuNP (13 nm in diameter), a radiolabeled oligonucleotide was used. The average number of attached oligonucleotides on each AuNP was estimated to be about 150 by measuring the radioactivity in the supernatant and on the AuNP pellet after centrifugation. The stability of this as-prepared AuNP-OD was then examined. To access the stabilities of DNA-modified AuNPs, the buffer concentration, pH (Tris-HCl, 20 mM, pH7.4) and NaCl concentrations (300 mM) were kept constant but the MgCl₂ concentration was optimized to achieve the highest MgCl₂ concentration at which DNA-modified AuNPs can just be stabilized. It was found that AuNP-OD were highly stable even at >500 mM MgCl₂, which is due to the fact that the highly negatively charged phosphate groups in DNA molecules can stabilize the AuNP against aggregation via electrostatic repulsion. However, such a highly stabilized AuNP-OD is not desirable since the assay must be conducted at a salt concentration at which AuNP-OD are not stable. In other words, the assay has to be performed at a MgCl₂ concentration higher than 500 mM, which is not suitable for the structure-switching aptamers: the DNA duplex between aptamer and its complement is highly stabilized at such a high salt concentration and thus this may hinder the aptamer structural switching from its complementary DNA to target molecules.

To tune the stability of AuNP-OD, Au—OD is briefly treated with 6-mercapto-1-hexanol (MCH) (5 μM) (room temperature (25° C.), 2 h). This ligand exchange process can remove both the nonspecifically adsorbed DNA and some of the thiol tethered DNA so that the concentration of DNA on each AuNP can be highly decreased. Indeed, radioactivity studies showed that after MCH treatment, the average number of oligonucleotides on each AuNP was about 97. One would expect, therefore, that the AuNP-OD after MCH exchange should have a lower stability against salt-induced aggregation. Indeed, it was found that AuNP-OD after MCH treatment can only be stabilized at salt concentrations less than 3 mM MgCl₂, 300 mM NaCl and 20 mM Tris-HCl (pH7.4). Higher MgCl₂ concentrations quickly lead to a solution color change (in 1 min) from red to purple.

It is noteworthy that the MCH treatment of AuNP-OD can significantly increase the hybridization efficiency between attached oligonucleotides on AuNPs and aptamers. This is because the dilution of oligonucleotide concentration on the AuNP surface and their conformational change after MCH treatment make them more accessible for the hybridization. Indeed, by using a radiolabeled aptamer, we found that the hybridization efficiency between attached oligonucleotide and aptamer for the MCH treated AuNPs was as high as about 90% whereas the hybridization efficiency for untreated AuNPs was only about 40%. This highly enhanced hybridization efficiency is essential in this study because it helps maximize the differences in electrophoretic properties between AuNP-OD and AuNP-OD-APT.

Compared to AuNP-OD, AuNP-OD-APT is significantly more stable. It is stable at salt concentrations as high as 35 mM MgCl₂, 300 mM NaCl, and 20 mM Tris-HCl (pH7.4); that is, there is no color change or significant spectra shift in UV-Vis spectra in 1 min, a time period designated for subsequent sensing experiments. In contrast, at the same salt concentration, AuNP-OD solution underwent an immediate color change and a red shift of the SPR band was observed. Accordingly, the subsequent adenosine sensing experiments were performed at 35 mM MgCl2, 300 mM NaCl, and 20 mM Tris-HCl (pH7.4).

In the presence of 1 mM adenosine, AuNP-OD-APT solution underwent an immediate color change from red to purple at 35 mM MgCl₂, 300 mM NaCl, and 20 mM Tris-HCl (pH7.4), which is corresponding to a red shift of SPR band in the UV-Vis spectra (as seen in FIG. 4). In contrast, the control experiments, where inosine, guanosine and cytosine were used, did not show any color change, which revealed the high specificity of the assay.

Kinetic studies of the color change in the presence of various amounts of adenosine were recorded by UV/Vis spectroscopy. To quantify the color change, the ratios of the absorbance at 700 and 525 nm (A700/A525) were re-plotted as a function of detection time. Clearly, faster rates of color change were obtained at higher adenosine concentrations as seen in FIG. 4. To further quantify the adenosine concentration, A700/A522 at 1 min after the addition of adenosine was re-plotted as a function of adenosine concentration. The detection limit of the assay under the investigated conditions was about 10 μM.

### Example 6

### DNA Modified AuNP Probes

Thiol-modified DNA (5.6 μL, 70 μM) was mixed with H₂O (25 μL) and then added to the AuNPs (50 μL, 13.4 nM). The solution was aged overnight and then added to phosphate buffer (other buffers, such as Tris-HCl, can also be applied) (10 mM, pH 7, NaCl, 0.1M) and aged for another 12 h. NaCl (0.3M) and phosphate (10 mM, pH 7) were then added to the mixture and the solution was allowed to stand for another 12 h. The resultant solution was centrifuged, and the particles were washed once with sodium phosphate buffer (200 μL, 0.3M, pH 7).

### Example 7

### Preparation of AuNP Aggregate Probe for DNase I Detection

A 100 μL solution contained two types of AuNPs modified with complementary DNA probes (1: 5′-GATCGACATGATGGCAAGCTTGTAGTGGATCGT10-SH; 2: 3′-T10CTAGCTGTACTACCGTTCGAACATCACCTAGC) (˜6 nM each Tris-HCl buffer (100 mM, pH 7.4) and NaCl (300 mM). The solution was heated at 70° C. for 2 min and allowed to cool at room temperature for 15 min. Then the mixture was stored at 4° C. for 6 h. A purple colored solution was observed, followed by AuNPs sedimentation. Just prior to spotting on various paper surfaces, aggregated AuNPs were spun down at 10,000 rpm for 10 min, then redispersed in the same buffer (50 μL) and 10 μL of the solution is spotted onto a piece of paper strip (Whatman filter paper #1) using a pipette.

### Discussion

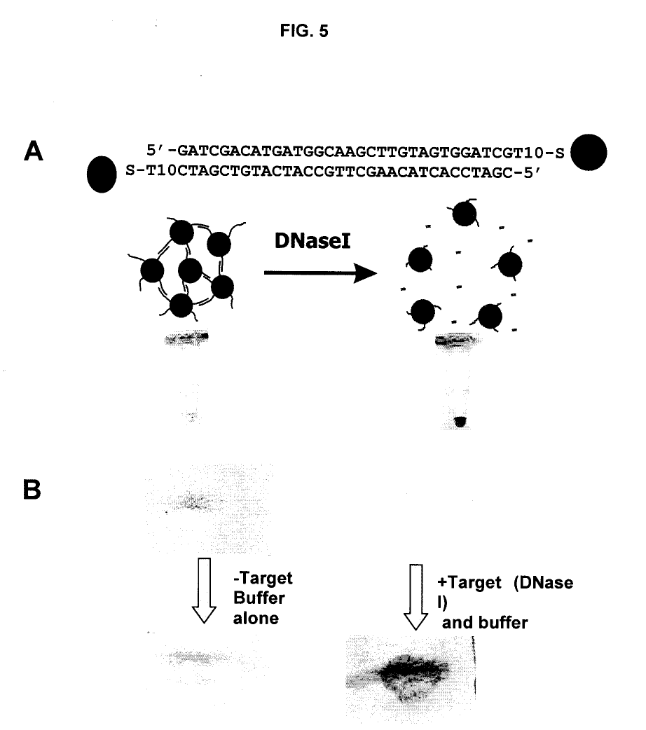

FIG. 5 shows a colorimetric AuNP/paper bioassay for protein/enzyme detection. (A) shows construction of AuNP aggregates which are prepared by DNA hybridization, between two complementary DNA probe-modified AuNPs. After centrifugation of these AuNP aggregates, the pellet shows a deep blue or even black color as seen in the left tube. The addition of DNase I, an enzyme that digests double-stranded DNA, breaks the aggregation of AuNPs into well-dispersed AuNPs which generates an intense red color as seen in the right tube. (B) shows paper-based assays for DNase I detection. These AuNP aggregates are spotted on paper (Whatman filter paper #1). Due to the optical nature of AuNP aggregate, the spots are colorless (or faint blue) on the paper of the left. However, upon the addition of DNase I, the DNase I dissociates the AuNP aggregates into well-dispersed AuNPs on the paper which is accompanied by the appearance of an intense red color as seen on the right hand paper.

### Example 8

### DNase I Detection on Polyvinyl Alcohol Coated Paper

In this example, DNase I is detected on paper that has been coated with polyvinyl alcohol. Whatman filter paper #1 was immersed in 1% polyvinyl alcohol (MW) for 1 min and dried in air. The same procedure as used in Example 7 was then repeated and a similar assay performance is observed.

### Discussion

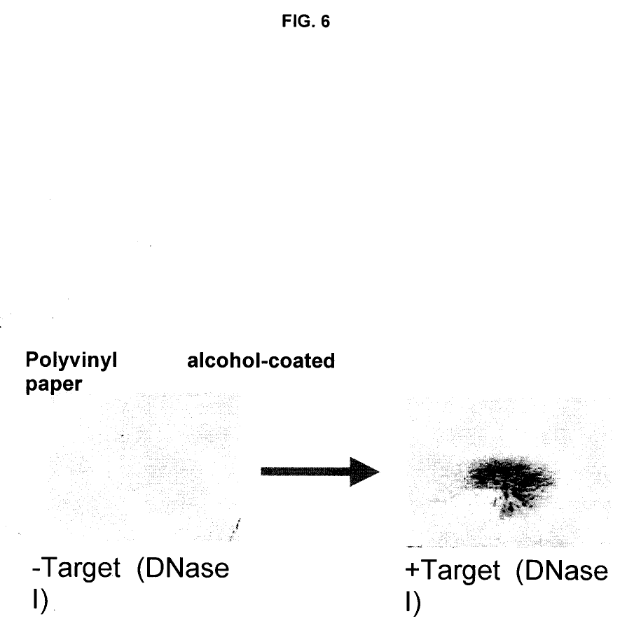

FIG. 6 shows colorimetric AuNP/paper bioassays using alcohol coated paper. AuNP aggregates prepared by DNA hybridization are spotted on polyvinyl alcohol-coated paper (Whatman filter paper #1). The addition of target DNase I sample showed an intense red color. It was found that polyvinyl alcohol-coated paper for the present system showed improved assay performance compared with non-coated paper.

### Example 9

### Preparation of AuNP Aggregate Probe for Adenosine Detection

Two types of AuNPs modified with complementary DNA probes (1: 5′-HS-CCCAGGTTCTCT; 2: 3′-A12TGAGTAGACACT) (˜6 nM each, 100 μL) were mixed with an adenosine aptamer crosslinker (ACTCATCTGTGAAGAGAACCTGGGGGAGTATTGCGGAGGAAGGT) in Tris-HCl buffer (100 mM, pH 7.4) with NaCl (300 mM). The solution was heated at 70° C. for 2 min and allowed to cool at room temperature for 15 min. The mixture was then stored at 4° C. for 6 h. A purple colored solution was observed and the AuNPs began to precipitate. Prior to spotting on paper, aggregated AuNPs were spun down at 10,000 rpm for 10 min, then redispersed in the same buffer (50 μL) and 10 μL of the solution is spotted onto a piece of paper strip (Whatman filter paper #1) using a pipette.

### Example 10

### Adenosine Detection Using Colorimetric AuNP/Paper Bioassay

Paper coated with AuNP aggregate probes were dried in air for 15 min. Sample solutions with adenosine (typically 1 mM) and without adenosine (control experiment), respectively, in a buffer) containing 10 mM Tris-HCl, pH=7.5, 5 mM MgCl₂) were directly applied to AuNP aggregate spots. A red color appears on the paper bearing adenosine after a few minutes whereas paper without adenosine did not show any color/color change.

### Discussion

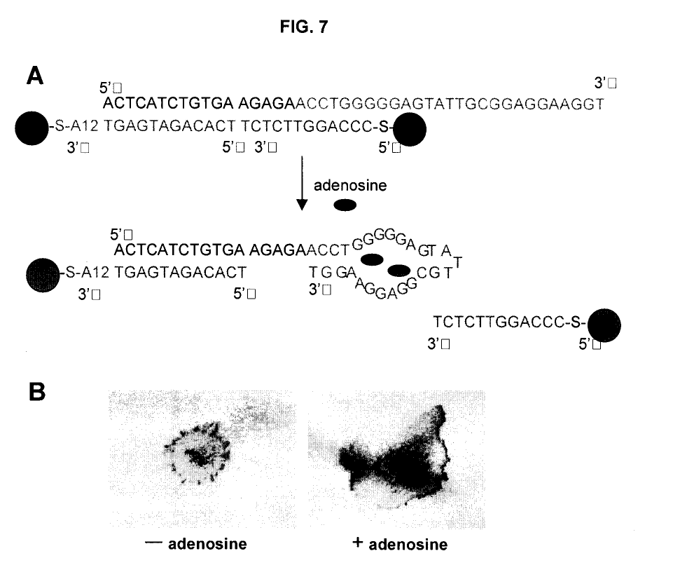

FIG. 7 shows a colorimetric AuNP/paper bioassay for small molecule detection. (A) shows the construction of an AuNP aggregate using adenosine DNA aptamer as a crosslinker that brings complementary DNA-modified AuNPs into aggregate. The addition of the target small molecule (adenosine) that binds to DNA aptamer dissociates AuNP aggregates into well-dispersed AuNPs. (B) shows the AuNP aggregate probes are spotted on paper (Whatman filter paper #1) which show a dark blue or black color. The addition of target small molecule (adenosine) turns on the intense red color due to the dissociation of the aggregated AuNPs into well-dispersed AuNPs.

### Example 11

### Colorimetric AuNP/Paper Bioassay for DNA Detection Using Paper Chromatography

A DNA probe-modified AuNPs probes (6 nM, 10 μL) (DNA sequence: 5′-HS-GTGACTGGACCC) was first prepared. The sample solution containing the target DNA (600 nM, 3′-CACTGACCTGGGGGAGTATTGCGGAGGAAGGT) was then incubated with DNA-probe modified AuNPs in a buffer containing Tris-HCl (50 mM), NaCl (100 mM), MgCl₂ (5 mM). The solution is heated up at 70° C. and allowed to cool at room temperature for 30 min. Then the DNA probe-modified AuNPs with and without DNA targets were spotted onto a piece of paper strip (Whatman filter paper #1).

After drying in air for 15 min, the paper strip was immersed in a developing buffer containing Tris-HCl (50 mM), NaCl (100 mM), Tween 20 (0.05%). As shown in FIG. 4, the AuNPs with target DNA appeared a red color because the attached additional target DNA strands provide extra stabilization whereas AuNPs without target DNA strands change color to blue.

### Discussion

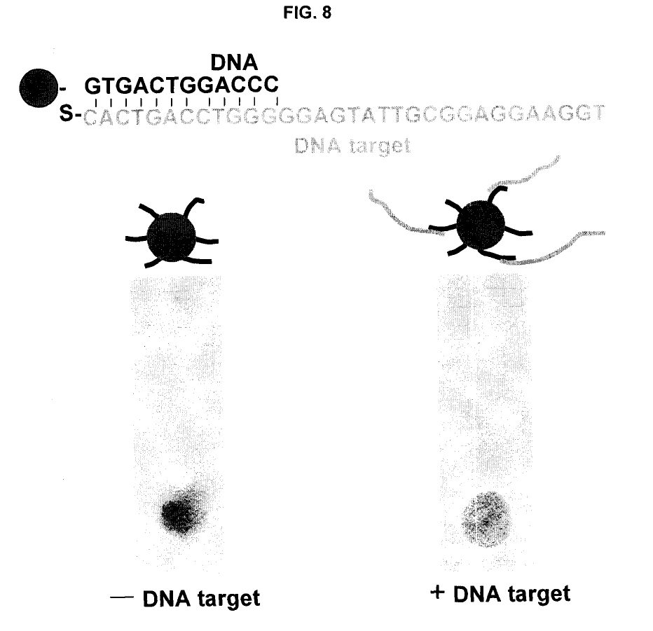

FIG. 8 shows a colorimetric AuNP/paper bioassay for nucleic acid detection. On the left, DNA probe-attached AuNPs change color from red to blue upon developing using paper chromatography platform: the migration of buffer brings AuNPs into aggregates. In contrast, when the target nucleic acid presents and binds to its complementary DNA probe on AuNPs (right), the AuNP color stays red on paper due to the extra stabilization effect provided by the target nucleic acid.

### Example 12

### Colorimetric AuNP/Paper Bioassay for ATP Detection Using Paper Chromatography

Complementary DNA-modified AuNPs (6 nM, 10 μL) (DNA sequence: 5′-HS-GTGACTGGACCC) was first prepared. ATP DNA aptamer (600 nM, 3′-CACTGACCTGGGGGAGTATTGCGGAGGAAGGT) was then incubated with complementary DNA-modified AuNPs in a buffer containing Tris-HCl (50 mM), NaCl (100 mM), MgCl₂ (5 mM). The solution was heated up at 70° C. and allowed to cool at room temperature for 30 min. The aptamer-modified AuNPs were then washed twice using the same buffer. Finally, the ATP aptamer-attached AuNPs (6 nM, 10 μL) were spotted onto a piece of paper strip (Whatman filter paper #1).

After drying in air for 15 min, the paper strip was immersed in a developing buffer containing Tris-HCl (50 mM), NaCl (100 mM), Tween 20 (0.05%) with or without the target molecule ATP (1 mM).

### Discussion

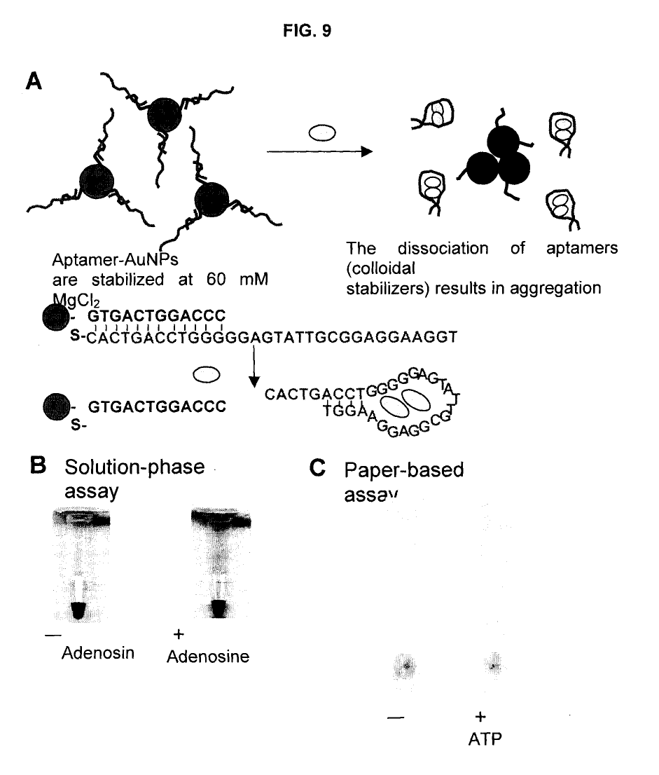

As shown in FIG. 9, in the presence of ATP, the AuNPs appears a blue color because the DNA aptamers that initially serve as AuNP stabilizers dissociate from AuNP to permit preferential binding of ATP—the resulting destabilized particles aggregate. By contrast, AuNP probes appear red in color if ATP is not present in the developing buffer. As shown in (A), the construction of AuNP probes is illustrated. Adenosine aptamer hybridizes with its short complementary DNA strand that is attached to AuNPs. The aptamer-modified AuNPs are highly stable at up to 60 mM MgCl₂ in buffer solution. The addition of a target molecule (adenosine) dissociates the aptamer from AuNP which destabilizes AuNP and therefore results in AuNP aggregation. This is shown in solution-based assays (B) and paper-based assays (C). With respect to paper-based assays, aptamer-modified AuNPs are spotted on paper. After drying, the paper is immersed into a developing buffer. When the developing buffer contains the target adenosine, the buffer migration on paper can dissociate aptamer from AuNPs and destabilize AuNPs. The association (aggregation) of AuNPs on paper therefore results in an intense red color signal.

### Example 13

### Colorimetric AuNP/Paper Bioassay for Hg²⁺ Detection Using Paper Chromatography

AuNPs attached with short complementary DNA were first prepared and then a DNA strand that binds to Hg²⁺ was hybridized to the surface (following the procedure in Example 12). Subsequently, the DNA-attached AuNP probes (6 nM, 10 μL) were spotted onto a piece of paper strip (Whatman filter paper #1). After drying in air for 15 min, the paper strip was immersed in a developing buffer containing Tris-HCl (50 mM), NaCl (100 mM), Tween 20 (0.05%), 30 mM MgCl₂, with or without target metal ion Hg²⁺ (500 μM), respectively.

### Discussion

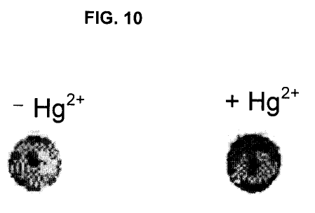

As shown in FIG. 10, the AuNPs in the presence of the target metal ion Hg²⁺ appear blue in color because the DNA molecules, served as AuNP stabilizers, dissociate from AuNP upon binding Hg²⁺, leading to particle aggregation. By contrast, AuNP probes appear red in color if Hg²⁺ is not included in the developing buffer. As with the ATP structuring DNA aptamer system, a structuring DNA strand that responds to Hg²⁺ are hybridized with the short complementary DNA strand that is attached with AuNPs. The DNA-AuNP probes are then spotted on paper. When the target metal ion (Hg²⁺) is included in the developing buffer, upon paper chromatography developing, Hg²⁺ dissociates DNA stands from AuNPs and therefore destabilizes AuNPs, which results in the AuNP aggregation on paper. The color therefore changes from red to blue on paper (right).

### Example 14

### Colorimetric AuNP/Paper Bioassay for Bacteria Detection Using Paper Chromatography

An E. coli (10 μL, concentration) aqueous solution was co-spotted with citrate ion-modified AuNPs (10 μL, 13 nM). After drying in air for 15 min, the paper strip was immersed in a water solution to develop.

### Discussion

In the absence of E. coli, the AuNPs in water migrate into close proximity along the paper strip, which generates a red-to-blue color change. By contrast, in the presence of E. coli, AuNP probes remain red in color, presumably due to the stabilization effect provided from E. coli. As seen in (A), bacteria stabilize AuNPs in solution (right tube) towards salt-induced aggregation. (B) shows a paper-chromatography based platform that is used for the detection of bacteria on paper. When target bacteria are co-spotted on paper (Whatman filter paper #1) together with citrate ion-capped AuNP probes, the bacteria-stabilized AuNPs (presumably due to electrostatic interactions) stay red color when a developing buffer is applied. By contrast, in the absence of target bacteria, AuNP probes change into a blue color due to the AuNP aggregation induced by flow of developing buffer.

### Example 15

### Effect of Incorporation of Surfactants

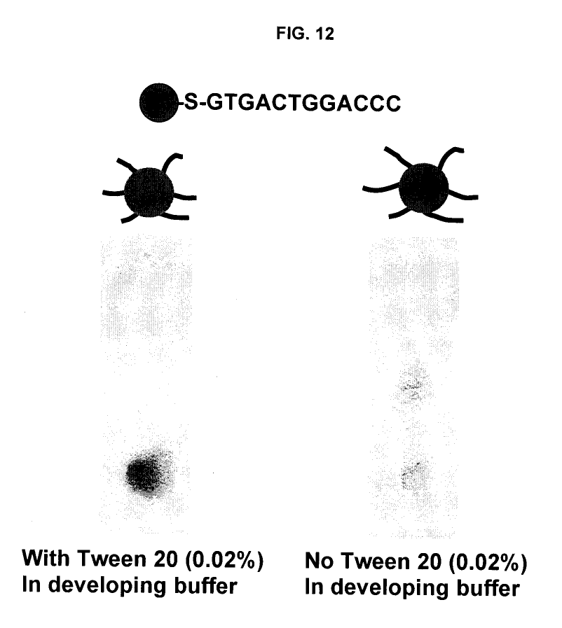

For paper chromatography-based paper bioassays, the performances can be improved by the incorporation of surfactants such as Tween 20 (0.05%). It helps analytes migration along paper and therefore facilitates the interaction of target analyte and AuNP probes (as seen in FIG. 12).

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term

1. A water-miscible AuNP, wherein the AuNP is stabilized with at least one capping ligand, the capping ligand having a AuNP binding domain and a charged domain.

2. The AuNP according to claim 1 wherein the diameter of the AuNP is about 1 nm to about 100 nm.

3. (canceled)

4. The AuNP according to claim 1 wherein the capping ligand is a nucleotide, a deoxynucleotide, a functionalized nucleotide, a nucleoside, an oligonucleotide, a functionalized oligonucleotide, a nucleic acid polymer, a thiol or an amine.

5. The AuNP according to claim 4 wherein the nucleotide is adenosine 5′-triphosphate (ATP), adenosine 5′-diphosphate (ADP), adenosine 5′-monophosphate (AMP), guanosine 5′-triphosphate (GTP), cytidine 5′-triphosphate (CTP), thymidine 5′-triphosphate (TTP), inosine 5′-triphosphate or uracil 5′-triphosphate.

6. (canceled)

7. The AuNP according to claim 4 wherein the oligonucleotide is a DNA or RNA oligonucleotide.

8. The AuNP according to claim 7 wherein the DNA or RNA oligonucleotide is able to hybridize an aptamer, wherein the aptamer can bind to a target.

9. The AuNP according to claim 8 wherein the target is a protein, an enzyme, nucleic acid, a small molecule, a metal ion, a bacteria or a pathogen.

10. The AuNP as claimed in claim 1, wherein the capping ligand is further functionalized.

11. The AuNP according to claim 10, wherein the capping ligand is chemically functionalized or enzymatically functionalized.

12. The AuNP as claimed in claim 1, wherein when the capping ligand of the AuNP is displaced, the AuNPs form aggregates.

13. A method for the production AuNPs comprising reacting a solution of a gold salt in a suitable solvent with a stabilizing capping ligand and a reducing agent, wherein the capping ligand has a binding domain and a charged domain.

14. The method according to claim 13, wherein the gold salt is HAuCl₄.

15. The method according to claim 13, wherein the HAuCl₄ is present in the suitable solvent in an amount of about 100 μM to about 100 mM.

16. (canceled)

17. The method according to claim 13, wherein the solvent is water.

18. The method according to claim 13, wherein the capping ligand is a nucleotide.

19. The method according to claim 18, wherein the nucleotide is adenosine 5′-triphosphate (ATP), adenosine 5′-diphosphate (ADP), adenosine 5′-monophosphate (AMP), adenosine, guanosine 5′-triphosphate (GTP), cytidine 5′-triphosphate (CTP), thymidine 5′-triphosphate (TTP), inosine 5′-triphosphate or uracil 5′-triphosphate.

20. (canceled)

21. The method according to claim 13, wherein the capping ligand is present in an amount of about 100 μM to about 100 mM.

22. (canceled)

23. The method according to claim 13, wherein the reducing agent is sodium borohydride (NaBH₄).

24. The method according to claim 23, wherein the sodium borohydride is present in an amount of about 100 μM to about 500 mM.

25. (canceled)

26. The method according to claim 13, wherein the reaction is performed at a temperature of about 10° C. to about 50° C.

27. (canceled)

28. The method according to claim 13, wherein the reaction is performed for a period of about 1 hour to about 5 hours.

29. (canceled)

30. The method according to claim 13, wherein the molar ratio of gold salt to the capping ligand ([gold salt]:[capping ligand]) is about 0.1 to about 10.

31. (canceled)

32. The method according to claim 30, wherein the size of the stable AuNPs is controlled by the molar ratios of gold salt, the capping ligand and the reducing agent.

33-35. (canceled)

36. A method of monitoring or detecting a substance or process that induces aggregation of AuNPs, or dissociation of aggregates of AuNPs, comprising contacting the process or substance with a AuNPs as claimed in claim 1 and observing or detecting a color change due to the aggregation or dissociation of the particles, wherein a color change is indicative of the substance or process.

37. A method of determining the presence or absence of an analyte comprising:
a) providing a solution of AuNPs, or aggregates of AuNPs, wherein the AuNPs are as defined in claim 1;
b) mixing the solution of AuNPs or aggregates with a biological, medical or environmental sample comprising an analyte; and
c) determining the presence or absence of the analyte.

38. The method of claim 37, wherein the presence or absence of the analyte in the solution is quantitatively determined by ultraviolet or visible light spectroscopy.

39. The method of claim 37, wherein the presence or absence of the analyte in the solution is qualitatively determined by a color change of the solution.

40. The method of according to claim 37, wherein the analyte is a protein, an enzyme, nucleic acid, a small molecule, a metal ion, a bacteria or a pathogen.

41. A method of determining the presence or absence of an analyte comprising:
a) providing AuNPs, or aggregates of AuNPs, on a paper substrate, wherein the AuNPs are as defined in claim 1;
b) providing a biological, medical or environmental sample comprising the analyte on the paper substrate; and
c) determining the presence or absence of the analyte.

42. The method of claim 41, wherein the presence or absence of the analyte in the sample is qualitatively determined by a color change on the paper substrate.

43. The method of claim 41, wherein the analyte is a protein, an enzyme, nucleic acid, a small molecule, a metal ion, a bacteria or a pathogen.

44. A method for the labeling or imaging a target compound in biological or medical samples comprising:
a) providing a solution AuNPs, or aggregates of AuNPs, wherein the AuNPs are as defined in claim 1;
b) contacting the solution of AuNPs with the biological sample containing a target compound; and
c) optionally imaging the target compound.

45. The method according to claim 44, wherein the target compound is imaged by microscopy.

46. The method of claim 44, wherein the target compound is a protein, an enzyme, nucleic acid, a small molecule, a metal ion, a bacteria or a pathogen.
